(19) United States
(12) Patent Application Publication
(10) Pub. No.: US 2026/0078257 A1
(43) Pub. Date: Mar. 19, 2026

(54) SYNTHESIS OF MULTIFUNCTIONAL BIOPOLYMER FROM COCONUT HUSK AND/OR COIR PITH POWDER AND ITS APPLICATION

(71) Applicant: REGERON, INC., Chuncheon-si (KR)

(72) Inventors: Sameer KUMAR, Dhule (IN); Shailesh S. CHALIKWAR, Dhule (KR); Kailas Kalicharan MORAVKAR, Chuncheon-si (KR)

(73) Assignee: REGERON, INC., Chuncheon-si (KR)

(21) Appl. No.: 18/923,164

(22) Filed: Oct. 22, 2024

(30) Foreign Application Priority Data
Sep. 19, 2024 (KR) 10-2024-0126708

(51) Int. Cl. C08L 97/00; A61L 15/28; A61L 15/40

(52) U.S. Cl. CPC C08L 97/005; A61L 15/28; A61L 15/40

(57) ABSTRACT

The present invention relates to the synthesis and production of biopolymer from natural waste—the husk of coconut and/or coir pith powder. More specifically it pertains to a novel multi-functional biopolymer obtained from the husk of coconut and/or coir pith powder, the fruit of Cocos nucifera with combinations of copolymer, crosslinker, and plasticizers for use in pharmaceutical, nutraceutical, cosmeceutical and leather industry. Lignin component from coconut husk was extracted, purified, studied for their physicochemical properties and processed into various biopolymers to be used as burn wounds dressings and leather substitutes.

### CROSS-REFERENCE TO RELATED APPLICATIONS

This application claims the benefit of Korean Application No. 10-2024-0126708, filed Sep. 19, 2024, in the Korean Intellectual Property Office. All disclosures of the document named above are incorporated herein by reference.

### TECHNICAL FIELD

The present invention relates to the extraction of lignin from coconut husk and/or coir pith powder. The extracted lignin is then subjected to purification and the physiochemical properties of the purified lignin are studied. The lignin is further processed with copolymers, crosslinkers, and plasticizers to create biopolymer sheets/films. These sheets/films exhibit multifunctional properties and have been found to be effective in wound dressing for severe burn accidents. The sheets/films have also been used for prototype production of a leather substitute material. The developed biopolymeric sheets/films have been characterized for their properties and have been found to be promising for various applications.

### BACKGROUND ART

Coconut husk and/or coir pith powder are versatile and eco-friendly materials that have gained popularity in various applications. These natural lignocellulosic fibers are obtained from the fruit of the Cocos nucifera, a tropical plant that belongs to the Arecaceae (Palmae) family. Their unique properties make them ideal for a wide range of uses, and their sustainable sourcing makes them an attractive option for those who prioritize environmental responsibility. Coconut husk and/or coir pith powder is produced globally with Indonesia, Philippines, India and Sri Lanka among the major contributors. Coconut fibre stands out as the most prominent fibrous byproduct of coconut cultivation, with an annual global production of at least 30 million tons. Most parts of coconut are used in food, fibre and other industries but the husk part is mainly used in crafts and decorations, and it remains vastly unused and its phyto-constituents remain in unexplored territory in the pharmaceutical field. The composition of coconut husk comprises 30% fibre and 70% pith, boasting high lignin and phenolic content according to Panyakaew and Fotios (2011). Van Dam et al. (2006) concluded that coconut husk comprised of 35% cellulose, 35% lignin and 17% hemicellulose (refer to FIG. 1). Thanks to its elevated lignin content, coconut fibre exhibits remarkable elasticity, durability, and resistance to decay.

Lignin is a highly beneficial complex organic polymer that plays a critical role in the structure of cell walls, particularly in wood and bark. It provides essential rigidity and is resistant to rotting, making it an excellent building material. In fact, among the lignocellulosic biomass, lignin is the second most abundant biopolymer after cellulose. Additionally, it forms strong and durable complexes with cellulose and hemicellulose, resulting in exceptional strength, flexibility, and high abrasion resistance, which is extremely useful in various industrial applications. Lignin consists of phenolic monomers, primarily coniferyl, sinapyl, and p-coumaryl alcohols, which are interconnected through various carbon-carbon and carbon-oxygen bonds, creating a highly branched and irregular structure. This complex network of aromatic rings and hydrocarbon chains contributes to lignin's strength and resilience, making it vital for plant growth and a valuable resource for numerous industrial applications.

Lignin, a highly complex organic polymer present in plant cell walls, is playing a pivotal role in numerous industries owing to its exceptional properties. It is an impressive biopolymer that plays a crucial role in enhancing the structural support, stiffness, and durability of plant tissues. Although cellulose and hemicellulose have traditionally received more attention in biomass utilization, lignin's unique and intricate nature has recently generated interest for its potential applications in sustainable materials, bioenergy, and various industrial domains. In the pulp and paper sector, lignin, previously considered a waste by-product, is now being utilized for the production of valuable chemicals like vanillin. It also serves as a sustainable adhesive in wood products, thereby reducing the reliance on synthetic resins. In the realm of bioplastics and composites, lignin enhances mechanical properties and contributes to biodegradability, particularly benefiting the production of automotive parts. Moreover, agriculture utilizes lignin derivatives as soil conditioners and controlled-release fertilizers, thereby improving usefulness and minimizing environmental impact. In the pharmaceutical industry, lignin's antioxidant and antimicrobial properties contribute to enhancing drug stability and delivery. Additionally, the cosmetic industry values lignin for its UV-protective qualities in sunscreens and anti-aging products. Furthermore, lignin can be converted into biofuels, thus supporting renewable energy initiatives. Our previously patent application of PCT/KR2022/019441 have expanded their application to include nutraceuticals and cosmetics. Finally, its unique chemical structure and properties can present more significant untapped potential in various new areas, particularly in the development of biopolymers.

The demand for novel biopolymer materials based on natural resources is on the rise in the leather, pharmaceutical, and cosmetic industries due to their sustainable nature, compliance with regulations, and enhanced functional properties. There is a growing push from consumers and governments for eco-friendly, biodegradable alternatives to traditional synthetic polymer materials to mitigate environmental impact and health risks. Biopolymers from natural sources like lignin offer distinctive benefits such as biocompatibility, antimicrobial properties, and customization, making them well-suited for various applications including sustainable leather alternatives, advanced drug delivery systems, and natural skincare products. This shift towards natural biopolymers fosters market diversification, encourages innovation, and aligns with the global trend towards more sustainable industrial practices.

Lignin-based biopolymers offer innovative and sustainable solutions across various industries. In the leather industry, they could serve as eco-friendly alternatives while maintaining high quality and durability. The pharmaceutical industry could benefit from the biocompatibility and functional properties of lignin, making it ideal for drug delivery systems, wound dressings, and other medical applications. In the cosmetic industry, integrating lignin-derived biopolymers into skincare products could provide natural ingredients with antioxidant and UV-protection benefits, thereby enhancing product efficacy and sustainability.

In the leather industry, animal leather has numerous disadvantageous issues including a slaughtering process that is carried out to obtain leather. In addition, there is a growing concern about the environmental pollution caused by the livestock industry's discharge of a large amount of waste and carbon dioxide for leather production. The use of toxic materials such as chromium in the process of manufacturing leather not only poses health risks to manufacturers but also contributes to environmental pollution when discharged with wastewater. On the other hand, lignin could improve leather treatment and finishing by enhancing texture, appearance, and durability, and provide natural antioxidant protection. Lignin could additionally serve as a natural dye and color stabilizer, and as a biodegradable adhesive, contributing to more eco-friendly manufacturing processes. Overall, lignin's use in leather production could promote sustainability and high-quality leather products.

Burn wounds are one of the most prevalent types of injury, trailing behind traffic accidents, falls, and physical violence. In the earlier part of the 20th century, the treatment options for burn patients were exceedingly limited, leading to frequent fatalities due to hypovolemic shock within the initial day of post-injury. However, the latter part of the 20th century witnessed remarkable advancements in regenerative medicine, burn therapy, and pharmacotherapy. Despite these strides, effectively managing burn wounds remains fraught with challenges. The recovery of burn wounds is a complex and prolonged journey, relying on diverse immune system-mediated repair mechanisms. It involves the restoration of disrupted tissue continuity resulting from an unforeseen event, like a fire. To attain full recovery, burn wounds undergo three successive stages: inflammation, the creation of granulation tissue (proliferation), and remodeling, which could potentially result in scar formation. The patient's immune system assumes a pivotal role in coordinating the entirety of the wound-healing process. During the initial phase of healing, injury is most prone to bacterial infection, which can exponentially lower the healing and might become fatal if left untreated. To overcome this problem, though many varieties of wound dressings have been tried such as surface dressings, hydrogels, hydrocolloids, foam, fibre, films, etc., they all turned out to be with proportionate setbacks such as biocompatibility issues, nature of dressing materials, ease of application, scraping of dressings, shape and size, activity on pathogens, drug loading capacity, and others. To tackle these issues, innovative biocompatible polymer development was needed.

Lignin-based biopolymer sheets and films could offer significant advantages for burns and wound dressings over traditional materials. Their natural antimicrobial and antioxidant properties could help prevent infections and reduce oxidative stresses, promoting faster healing. These biopolymers could form hydrogels, creating a moist healing environment while managing excess exudate, thereby facilitating cell migration, tissue regeneration, and the removal of necrotic tissue. These processes are crucial for optimal wound healing outcomes. Their biocompatibility also reduces the risk of allergic reactions and minimizes potential of irritations. Besides, they could be used to create flexible, durable dressings that not only protect wounds from contaminants, but also allow for controlled drug delivery. In other words, lignin-based biopolymers have the potential to be engineered for delivering therapeutic agents such as antimicrobial agents or growth factors, directly to the wound site in a controlled manner, ensuring sustained treatment. This targeted drug delivery approach could aim to enhance treatment efficacy while minimizing systemic side effects. Moreover, they are biodegradable and cost-effective, making them aligned with environmentally friendly, sustainable healthcare practices. Overall, lignin-based biopolymer dressings offer attractive options for versatile and effective solutions for advanced wound care, contributing to improved patient outcomes and reduced environmental impact.

The present invention discloses the development of various lignin-based biopolymer blends using extracted lignin from coconut husk and/or coir pith powder to prepare biopolymer sheets and/or films for burn healing/wound dressings and as leather substitute materials. The lignin polymer is produced through the oxidative radical polymerization of three major p-hydroxycinnamyl alcohols, known as monolignols, namely, p-coumaryl alcohol, coniferyl alcohol, and sinapyl alcohol. These alcohols are responsible for the formation of p-hydroxyphenyl (H), guaiacyl (G), and syringyl(S) units, which are incorporated into the lignin polymer.

Hardwoods consist of syringyl(S) and guaiacyl (G) units, while softwoods primarily consist of guaiacyl (G) units with minor amounts of p-hydroxyphenyl (H) units (FIG. 2).

### DISCLOSURE

### Technical Problem

The principle objective of the present invention is to extract the lignin from the coconut husk and/or coir pith powder obtained from a fruit of the Cocos nucifera, a tropical plant that belongs to the Arecaceae (Palmae) family to develop a novel natural biocompatible multi-functional biopolymer which can be used in pharmaceutical, nutraceutical, cosmeceutical, cosmetic and/or leather industry applications.

Another objective of the present invention is to extract the Lignin component from coconut husk and/or coir pith powder, purify, and study for its physicochemical properties and process it into various biopolymer sheets/films/gels using other additional copolymers, crosslinkers and plasticizers.

It is another objective of the present invention to develop a biopolymeric sheet/film/gel for victims of severe burn accidents, whereas this protective wound dressing layer is made up with combination of 1) lignin extracted from coconut husk and/or coir pith powder, and 2) α-lactalbumin extracted from whey protein isolate, and 3) additional other copolymers, crosslinkers, and plasticizers. Due to its anti-microbial, anti-oxidant, and moist-keeping properties, lignin biopolymer has a potential for wound dressing in severe burn accidents to rejuvenate new growths which replace damaged tissue on the burn wounds, especially when combined with API's like recombinant human growth factors. It can be further used in healing and restoration of a disrupted tissue through diverse immune system-mediated repair mechanisms and new growths-accompanied regeneration and replacement of the damaged burn tissue.

Another objective of the present invention is to provide leather substitute material which can be converted into leather (non-animal source) using leather reinforcement material obtained from the fibres of coconut husk and/or coir pith powder so as to exhibit excellent wear resistance and flexibility, particularly as a leather substitute.

### Technical Solution

As agricultural wastes, coconut husk and/or coir pith powder were proven to be rich sources of lignin as well as cellulose. Lignin solubility improves depending on methods of extraction and drying as well as adjustments in pH and temperature. Lignin was successfully converted into multifunctional biopolymeric sheets/films/gels using various additional copolymers, crosslinkers and plasticizers, although the effect of each additive kind was studied at a basic level. This invention developed multifunctional biopolymeric sheets/films/gels and studied their application effects with regard to manufacturing processes and resultant qualities of wound dressing and leather substitute materials, respectively.

Lignin is a remarkable organic polymer with a diverse range of chemical constituents that make it exceptionally well-suited for burn and wound healing dressings. Its phenolic compounds possess natural antimicrobial and antioxidant properties, effectively preventing infections and reducing oxidative stress to facilitate healing. The primary monolignols-coniferyl alcohol, sinapyl alcohol, and coumaryl alcohol-play a pivotal role in maintaining the structural integrity and anti-microbial activity of lignin, supporting tissue repair. Methoxy groups enhance chemical reactivity, allowing for the development of customized derivatives for improved moisture retention and controlled drug release. Moreover, carboxylic acid groups increase hydrophilicity, leading to formation of hydrogels that sustain a moist healing environment and enhance the therapeutic agents-binding capacity. Additionally, quinones introduce redox properties that aid in managing infections and inflammation, while lignin-carbohydrate complexes promote biocompatibility, rendering lignin a versatile and highly effective material for advanced wound care solutions.

On the other hand, alpha-lactalbumin was reported to play a superior role in promoting regeneration of new skin on burn wounds through stimulating fibroblast proliferation and adhesion and promoting vascularization by way of increasing local serotonin concentration, resulting in faster healing at burn wound sites. It supports supply of essential amino acids, especially a highest level of tryptophan among other nutrients, necessary for protein synthesis and tissue repair. Moreover, it helps regulate the immune response, minimizing excessive inflammation and controlling cytokine production, and boost collagen synthesis for structural integrity. When added into the burn wound dressings incorporating lignin, alpha-lactalbumin also helps create even better moist wound environment essential for cell migration and tissue formation, while lignin's antioxidant properties alleviate oxidative stresses, further facilitating efficient skin regeneration.

In addition to above uses for burn wound dressings, alpha-lactalbumin could be incorporated into multifunctional lignin biopolymer that constitutes prototype leather substitute materials derived from the fibres of coconut husk and/or coir pith powder, with or without additional leather reinforcement materials. The resulting leather substitutes could provide excellent wear resistance from lignin and softened wrapping flexibility from alpha-lactalbumin, if provided ideal compositions.

More specifically, the present invention relates to a natural biocompatible multi-functional biopolymer comprising; a) Lignin extracted from the coconut husk and/or coir pith powder obtained from a fruit of the Cocos nucifera, a tropical plant that belongs to the Arecaceae (Palmae) family; b) copolymers; c) crosslinkers; d) plasticizers; and e) at least one active agent.

In the other embodiment, the disclosure relates to a novel natural biocompatible multi-functional biopolymer wherein at least one copolymer is selected from either a natural polymer or a water-soluble synthetic polymer. Natural polymers are such as hyaluronic acid and its derivatives, starch, modified starch, alginates, chitosan, chitin, natural gums, proteins such as gelatin, collagen, casein, zein, gluten, soy protein isolate, and whey protein isolate, and land plant extracts such as pectin, and combinations thereof. Water-soluble synthetic copolymers include cross-linked polyacrylic acid chains, polyvinyl pyrrolidone, polyvinyl alcohol, modified cellulose ethers such as hydroxypropyl methylcellulose, hydroxyethyl cellulose, hydroxypropyl cellulose, carboxymethyl cellulose, and ion-exchange resins, and combinations thereof. The effective amount of copolymer ranges from approximately 10% to 90% dry weight, with a preference for 30% to 70% dry weight, depending on the application of the developed biocompatible multi-functional biopolymer.

In the other embodiment, the disclosure relates to a novel natural biocompatible multi-functional biopolymer wherein at least one crosslinker is selected from glutaraldehyde, formaldehyde, polyethylene glycol (PEG) and its derivatives, genipin, dextran, polyethyleneimine (PEI), ethylenediamine (EDA), and sugar alcohols/polyols like erythritol, xylitol, maltitol, mannitol, lactitol, polyglycitol, isomalt, sorbitol, glycerols, and combinations thereof, to promote crosslinking chemical reactions. The effective amount of crosslinkers ranges from approximately 1% to 10% dry weight, with a preference for 2% to 5% dry weight, depending on the application of the developed biocompatible multi-functional biopolymer.

In the other embodiment, the disclosure relates to a novel natural biocompatible multi-functional biopolymer wherein at least one plasticizer is selected from sucrose esters derivatives, glycerin, propylene glycol, polyethylene glycol, magnesium stearate, tallow, neatsfoot oil, lanolin, mineral oils, silicon oils, jojoba oils, castor oil, coconut oil, whale oil, squalene, grape seed oil, safflower oil, canola oil, almond oil, etc., and combinations thereof. The effective amount of plasticizers ranges from approximately 0.5% to 10% dry weight, with a preference for 1% to 3% dry weight, depending on the application of the developed biocompatible multi-functional biopolymer.

In the other embodiment, the disclosure relates to a novel natural biocompatible multi-functional biopolymer wherein optionally an emulsifying agent is selected from xanthan gum, lanolin, sodium alginate, lecithin, casein, whey protein, pectin, ammonium phosphatides, acacia, and oleylamine. The effective amount ranges approximately from 0.5-5% (w/w), depending on the application of the developed biocompatible multi-functional biopolymer.

In the other embodiment, the disclosure relates to a novel natural biocompatible multi-functional biopolymer wherein optionally an emulsifying agent is selected from xanthan gum, lanolin, sodium alginate, lecithin, casein, whey protein, pectin, ammonium phosphatides, acacia, and oleylamine.

In the other embodiment, the disclosure relates to a novel natural biocompatible multi-functional biopolymer where it is used as a wound dressing or a part of the wound dressing on severe burn wounds of a patient to rejuvenate new tissue growths and replace damaged tissue on the burn wounds, wherein at least one active agent may be loaded from turmeric extract (curcumin), honey, green tea extract, gotu kola extract (Centella asiatica), α-lactalbumin, whey protein isolate, comfrey extract, tea tree oil, lavender oil, chamomile extract, calendula extract, aloe vera gel, silver sulfadiazine, bacitracin, hyaluronic acid, collagen, vitamin E, allantoin, sphingosine-/phytosphingosine-1-phosphate, and dexpanthenol (vitamin B5), and combinations thereof. The effective amount of at least one active agent ranges from approximately 0.01% to 50% dry weight, with a preference for 1% to 20% dry weight, depending on the application and severity of the burn wounds.

In the other embodiment, the disclosure relates to a novel natural biocompatible multi-functional biopolymer where it is used as a wound dressing or a part of the wound dressing on severe burn wounds of a patient to rejuvenate new tissue growths and replace damaged tissue on the burn wounds, wherein at least one active agent may be loaded from human growth factors and/or cytokines such as Epidermal growth factor (EGF), Growth hormone/Somatotropin (hGH/STH), Vasoactive intestinal peptide (VIP), Fibroblast growth factor family proteins, i.e., Fibroblast growth factor-1 (FGF1), Fibroblast growth factor-2 (FGF2), Fibroblast growth factor-7/Keratinocyte growth factor (FGF7; KGF), Fibroblast growth factor-10/Keratinocyte growth factor-2 (FGF10; KGF2), Fibroblast growth factor-19 (FGF19), Fibroblast growth factor-21 (FGF21), Fibroblast growth factor-23 (FGF23), etc., Vascular endothelial growth factor (VEGF), Thymosin beta-4 (TMS), Stem cell factor/Steel factor (SCF), Platelet-derived growth factor (PDGF), Prolactin (PRL), Placental lactogen (hPL), Nerve growth factor (NGF), Brain-derived neurotrophic factor (BDNF), Glial cell-derived neurotrophic factor (GDNF), Ciliary neurotrophic factor (CNTF), Interleukin-1 receptor antagonist (IL1Ra), Interleukin family proteins, i.e., Interleukin-2 (IL-2), Interleukin-4 (IL-4), Interleukin-13 (IL-13), Interleukin-17 (IL-17), Interleukin-18 (IL-18), etc., Adiponectin (ApN), TSLP (Thymic stromal lymphopoietin), Bone morphogenetic protein family proteins, i.e., BMP-2, BMP-4, BMP-7, etc., Cluster of differentiation-34 (CD34), Desmoplakin (Dsp), Erythropoietin (EPO), Erythroid Differentiation Regulator 1 (ERDR1), Fibronectin, Follicle stimulating hormone (FSH), Luteinizing hormone (LH), human Chorionic gonadotropin (hCG), Follistatin (FST), Growth differentiation factor family proteins, i.e., Growth differentiation factor 11/Bone morphogenetic protein-11 (GDF11/BMP-11)), Growth differentiation factor 15 (GDF15), etc., Irisin, Kisspeptins, Klotho, Matrikines (extracellular matrix-derived peptides), Scube3 (signal peptide, CUB and EGF-like domain-containing protein 3), Osteopontin, Oxytocin, Pregnancy associated plasma protein-A (PAPP-A), Platelet factor 4 (PF4), Reelin, Stem cells antigen-1 (Sca-1), Stanniocalcin-1 (STC1), Stanniocalcin-2 (STC2), Transforming growth factor (TGF) family proteins, i.e., TGF-alpha, TGF beta-1, TGF beta-2, TGF beta-3, Thyrotropin-releasing hormone (TRH), Thyroid-stimulating hormone (TSH), Heat shock protein family proteins, i.e., HSP90A and its derivatives, etc., and their combinations thereof. The effective amount of at least one or more active agents ranges from approximately 0.000001% to 1% dry weight, with a preference for 0.00001% to 0.1%, with a more preference for 0.00005% to 0.005%, depending on the application and severity of the burn wounds.

In the other embodiment, the disclosure relates to a novel natural biocompatible multi-functional biopolymer wherein the combination of lignin and α-lactalbumin has demonstrated to make an efficacious wound dressings for severe burn wounds, wound healing, reducing scarring and hastening re-epithelization and to rejuvenate new tissue growths and replace damaged burn wound tissues.

In the other embodiment, the disclosure relates to a novel natural biocompatible multi-functional biopolymer where it is used to develop a leather substitute or a part of the leather substitute, wherein a prototype of the leather substitute with or without using either α-lactalbumin or leather reinforcement materials obtained from the fibres of coconut husk and/or coir pith powder, exhibits excellent wear resistance and flexibility of those of the natural leather.

### DESCRIPTION OF DRAWINGS

FIG. 1: Depicts a) percent distribution of components in coconut husk; and b) percent distribution of lignin in different plant material.

FIG. 2: Depicts the structures of lignin constituents.

FIG. 3a: Depicts the extraction of lignin from coconut husk.

FIG. 3b: Depicts the isolation of α-lactalbumin from whey protein.

FIG. 4: Depicts Zeta potential and particle size of lignin.

FIG. 5: Depicts ATR-IR graph of extracted lignin.

FIG. 6: Depicts X-ray diffractogram (XRD) of extracted lignin.

FIG. 7: Depicts Differential scanning calorimetric (DSC) graph of extracted lignin.

FIG. 8: Depicts Polarized light microscopy graph of Ferric Chloride Test.

FIG. 9: Depicts the lignin-based biopolymer films-HLG(F1), XGPG(F2), and PXLG(F3).

FIG. 10: Depicts the thickness of the films.

FIG. 11: Depicts the folding endurance of lignin-based biopolymer films.

FIG. 12: Depicts Texture and elastic modulus graph of lignin-based biopolymer films-HLG(F1), XGPG(F2), and PXLG(F3).

FIG. 13: Depicts Tensile strength and elastic modulus analysis of lignin-based biopolymer films.

FIG. 14: Depicts the degrees of swelling of lignin-based biopolymer films.

FIG. 15: Depicts the % water retention of lignin-based biopolymer films.

FIG. 16: Depicts the water vapour transmission of lignin-based biopolymer films.

FIG. 17a: Depicts film showing photolytic degradation of excess glutaraldehyde as CO₂ at 2360 cm⁻¹.

FIG. 17b: Depicts IR spectra of HLG film.

FIG. 17c: Depicts IR spectra of PXLG film.

FIG. 17d: Depicts IR spectra of XGPG film.

FIG. 18: Depicts XRD analysis graph of lignin-based biopolymer films.

FIG. 19: Depicts DSC overlay of α-lactalbumin loaded XGPG film.

FIG. 20: Depicts SEM image of lignin-based biopolymer XGPG (F2) film.

FIG. 21: Depicts the histopathology of the tissue samples-(Normal group-no injury induced and kept for reference; Negative control was given injury but no treatment was given; Blank film group received blank lignin-based biopolymer film lacked alpha-lactalbumin; Standard group were also given injury was treated with marketed silver sulfadiazine cream: Treatment group were given injury and they were treated with films which contain alpha-lactalbumin).

FIG. 22: Depicts a prototype of animal leather substitutes from lignin-based biopolymer.

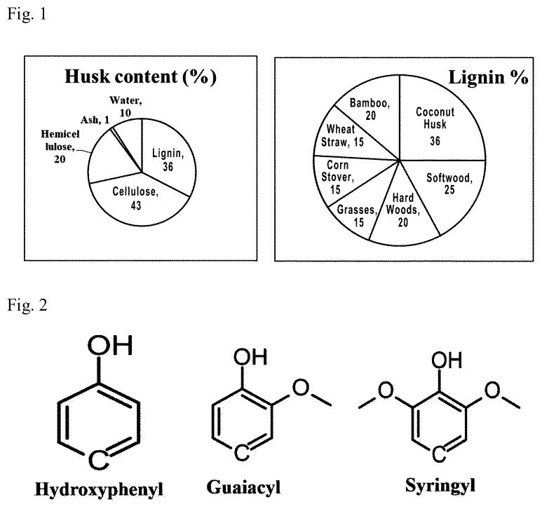

### BEST MODE

Those skilled in the art will be aware that the present disclosure is subject to variations and modifications other than those specifically described. It is to be understood that the present disclosure includes all such variations and modifications. The disclosure also includes all such steps, features, compositions, and compounds referred to or indicated in this specification, individually or collectively, and any and all combinations of any or more of such steps or features.

For convenience, before further description of the present disclosure, certain terms employed in the specification, and examples are collected here. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”.

Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

The term “including” is used to mean “including but not limited to”. “Including” and “including but not limited to” are used interchangeably.

Ratios, concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited.

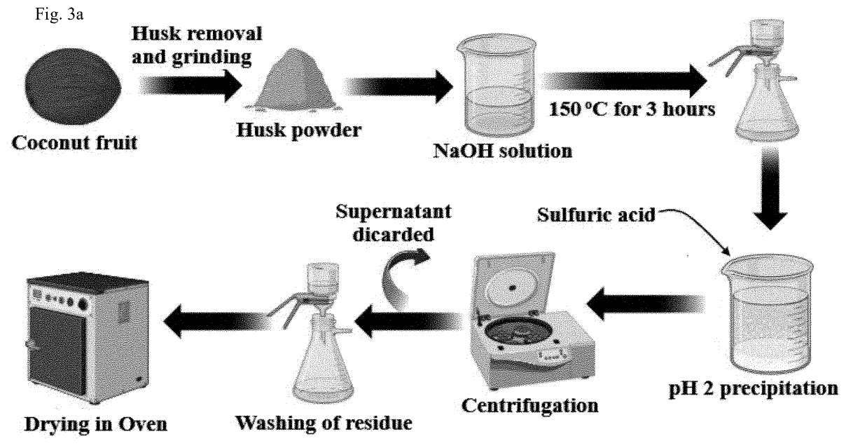

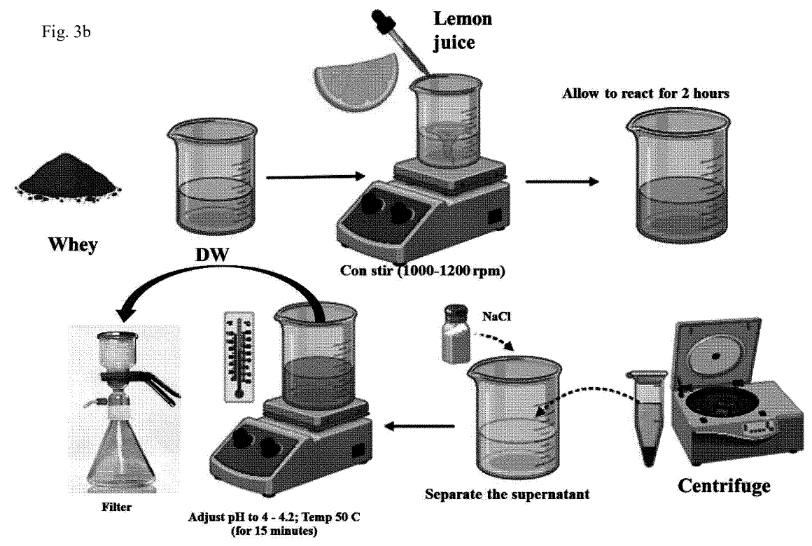

In an embodiment of the present disclosure, there is provided a process for the extraction and purification of lignin from coconut husk and/or coir pith powder comprising: 1) The Kraft pulping method was utilized for the extraction of lignin from coconut husk; 2) A solution was made in 200 ml of 12.5% w/v aq. NaOH solution by adding husk powder in the range of 10-80 g, more preferably 20-40 g of husk powder, and kept in an autoclave (Lequitron) at 130° C. for 3 hours; 3) The solution of NaOH containing dissolved lignin was then filtered through muslin cloth using a vacuum filter, and residue/filter-cake containing delignified mass was kept separately, and the filtrate (black liquor) was collected and kept in a beaker; 4) The collected black liquor was then treated with concentrated sulfuric acid until it reached pH 2 where precipitated lignin became clearly visible, and the solution was left to settle for 4 hours (the lignin sediments were at the bottom while acidic liquid formed a separate layer on top); 5) the liquid portion was carefully discarded, and the remaining solution was then centrifuged at 10,000 RPM for 10 minutes, and the supernatant was discarded and the residue in the form of lignin was obtained; 6) This lignin was further washed with distilled water and passed through a vacuum filter using filter paper (4.5 um pore size, Nylon 66); 7) The collected lignin was dried in a hot air oven overnight at 60° C. and later crushed using a mortar and pestle to a fine powder form and stored in a polybag (FIG. 3a).

In an embodiment of the present disclosure herein illustrates development of a novel natural biocompatible multi-functional biopolymer comprising extracted lignin component from coconut husk and/or coir pith powder, copolymers, crosslinkers, and plasticizers.

In an embodiment of the present disclosure herein illustrates development of a novel natural biocompatible multi-functional biopolymer comprising extracted lignin component from coconut husk and/or coir pith powder, copolymers, crosslinkers, and plasticizers, wherein the copolymer can be either synthetic or natural. Some examples of natural polymers are hyaluronic acid and its derivatives, starch, modified starch, alginates, chitosan, chitin, natural gums, proteins such as gelatin, collagen, casein, zein, gluten, soy protein isolate, and whey protein isolate, and land plant extracts such as pectin, and combinations thereof. Water-soluble synthetic copolymers include cross-linked polyacrylic acid chains, polyvinyl pyrrolidone, polyvinyl alcohol, modified cellulose ethers such as hydroxypropyl methyl cellulose, hydroxyethyl cellulose, hydroxypropyl cellulose, carboxymethyl cellulose, and ion-exchange resins. The effective amount of copolymer ranges from approximately 10% to 90% dry weight, with a preference for 30% to 70%, depending on the application of the developed biocompatible multi-functional biopolymer.

The term ‘copolymer’ is a material which forms a network structure by combining two or more polymers, where at least one polymer is synthesized or crosslinked in the immediate presence of the other(s). These different polymers and/or copolymers are physically entangled with or within each other, creating a single, intertwined network, resulting in enhanced mechanical and physical properties ideal for various applications such as biomedical adhesives and impact-resistant materials. While the polymers and copolymers may not necessarily be covalently bonded to each other (i.e., may be covalently bonded by way of crosslinkers, for example), they form a cohesive structure that cannot be separated without breaking some chemical bonds connecting them.

In an embodiment of the present disclosure herein illustrates development of a novel natural biocompatible multi-functional biopolymer comprising extracted lignin component from coconut husk and/or coir pith powder, copolymers, crosslinkers, and plasticizers, wherein the crosslinkers may be selected from glutaraldehyde, formaldehyde, polyethylene glycol (PEG) and its derivatives, genipin, dextran, polyethyleneimine (PEI), ethylenediamine (EDA), and sugar alcohols/polyols like erythritol, xylitol, maltitol, mannitol, lactitol, polyglycitol, isomalt, sorbitol, glycerols, and combinations thereof, to promote crosslinking chemical reactions. The effective amount of crosslinkers ranges from approximately 1% to 10% dry weight, with a preference for 2% to 5%, depending on the application of the developed biocompatible multi-functional biopolymer.

In an embodiment of the present disclosure herein illustrates development of a novel natural biocompatible multi-functional biopolymer comprising extracted lignin component from coconut husk and/or coir pith powder, copolymers, crosslinkers, and plasticizers, wherein the plasticizers may be selected from sucrose ester derivatives, glycerin, propylene glycol, polyethylene glycol, magnesium stearate, tallow, neatsfoot oil, lanolin, mineral oils, silicone oils, jojoba oils, castor oil, coconut oil, whale oil, squalene, grape seed oil, safflower oil, canola oil, almond oil, etc., and combinations thereof. The effective amount of plasticizers ranges from approximately 0.5% to 10% dry weight, with a preference for 1% to 3%, depending on the application of the developed biocompatible multi-functional biopolymer.

In an embodiment of the present disclosure herein illustrates development of a novel natural biocompatible multi-functional biopolymer comprising extracted lignin component from coconut husk and/or coir pith powder, copolymers, crosslinkers, and plasticizers, wherein optionally an emulsifying agent is selected from xanthan gum, lanolin, sodium alginate, lecithin, casein, whey protein, pectin, ammonium phosphatides, acacia, and oleylamine. The effective amount ranges approximately from 0.5 to 5% (w/w), depending on the application of the developed biocompatible multi-functional biopolymer.

In an embodiment of the present disclosure, there is provided development of a novel natural biocompatible multi-functional biopolymer, which was successfully studied for wound dressings on severe burn accidents to rejuvenate new growths that replaced damaged tissues on burn wounds, wherein at least one or more active agents may be loaded from Turmeric extract (curcumin), Honey, Green tea extract, Gotu kola extract (Centella asiatica), α-Lactalbumin, Whey protein isolate, Comfrey extract, Tea tree oil, Lavender oil, Chamomile extract, Calendula extract, Aloe vera gel, Silver sulfadiazine, Bacitracin, Hyaluronic acid, Collagen, Vitamin E, Allantoin, Sphingosine-/Phytosphingosine-1-phosphate, and Dexpanthenol (Vitamin B5). The effective amount of at least one or more active agents ranges from approximately 0.01% to 50% dry weight, with a preference for 1% to 20%, depending on the application and severity of the burn wounds.

In an embodiment of the present disclosure, there is provided development of a novel natural biocompatible multi-functional biopolymer, which was successfully studied for wound dressings on severe burn accidents to rejuvenate new growths that replaced damaged tissues on burn wounds, wherein at least one or more active agents may be loaded from human growth factors and/or cytokines such as Epidermal growth factor (EGF), Growth hormone/Somatotropin (hGH/STH), Vasoactive intestinal peptide (VIP), Fibroblast growth factor family proteins, i.e., Fibroblast growth factor-1 (FGF1), Fibroblast growth factor-2 (FGF2), Fibroblast growth factor-7/Keratinocyte growth factor (FGF7; KGF), Fibroblast growth factor-10/Keratinocyte growth factor-2 (FGF10; KGF2), Fibroblast growth factor-19 (FGF19), Fibroblast growth factor-21 (FGF21), Fibroblast growth factor-23 (FGF23), etc., Vascular endothelial growth factor (VEGF), Thymosin beta-4 (TMS), Stem cell factor/Steel factor (SCF), Platelet-derived growth factor (PDGF), Prolactin (PRL), Placental lactogen (hPL), Nerve growth factor (NGF), Brain-derived neurotrophic factor (BDNF), Glial cell-derived neurotrophic factor (GDNF), Ciliary neurotrophic factor (CNTF), Interleukin-1 receptor antagonist (IL1Ra), Interleukin family proteins, i.e., Interleukin-2 (IL-2), Interleukin-4 (IL-4), Interleukin-13 (IL-13), Interleukin-17 (IL-17), Interleukin-18 (IL-18), etc., Adiponectin (ApN), TSLP (Thymic stromal lymphopoietin), Bone morphogenetic protein family proteins, i.e., BMP-2, BMP-4, BMP-7, etc., Cluster of differentiation-34 (CD34), Desmoplakin (Dsp), Erythropoietin (EPO), Erythroid Differentiation Regulator 1 (ERDR1), Fibronectin, Follicle stimulating hormone (FSH), Luteinizing hormone (LH), human Chorionic gonadotropin (hCG), Follistatin (FST), Growth differentiation factor family proteins, i.e., Growth differentiation factor 11/Bone morphogenetic protein-11 (GDF11/BMP-11)), Growth differentiation factor 15 (GDF15), etc., Irisin, Kisspeptins, Klotho, Matrikines (extracellular matrix-derived peptides), Scube3 (signal peptide, CUB and EGF-like domain-containing protein 3), Osteopontin, Oxytocin, Pregnancy associated plasma protein-A (PAPP-A), Platelet factor 4 (PF4), Reelin, Stem cells antigen-1 (Sca-1), Stanniocalcin-1 (STC1), Stanniocalcin-2 (STC2), Transforming growth factor (TGF) family proteins, i.e., TGF-alpha, TGF beta-1, TGF beta-2, TGF beta-3, Thyrotropin-releasing hormone (TRH), Thyroid-stimulating hormone (TSH), Heat shock protein family proteins, i.e., HSP90A and its derivatives, etc., and their combinations thereof. The effective amount of at least one or more active agents ranges from approximately 0.000001% to 1% dry weight, with a preference for 0.00001% to 0.1%, with a more preference for 0.00005% to 0.005%, depending on the application and severity of the burn wounds.

In an embodiment of the present disclosure, there is provided a process for the isolation of α-lactalbumin from whey protein (purchased from Suzhou Greenway Biotech Co., Ltd, China) comprising processes of extracting α-lactalbumin from whey protein isolate (90%) and removing Casein protein, which is responsible for the white color of milk, via centrifugation. Subsequently, NaCl was utilized as a washing and osmotic agent. The protein-bound Ca++ ions were made released from α-lactalbumin by reducing the pH to 4.2, aided by Citric acid monohydrate (40 mM/L). This resulted in conversion of Ca++-bound α-lactalbumin to a molten globule apo-form devoid of Ca++, which could be collected through a vacuum filter. Restoration of the pH balance caused the apo-form to return to its native form in the presence of Ca++. This method resulted in the effective and efficient extraction of α-lactalbumin (FIG. 3b).

In an embodiment of the present disclosure, there is provided development of a novel natural biocompatible multi-functional biopolymer, which was successfully studied for wound dressing on severe burn accidents to rejuvenate new growths that replaced damaged tissues on burn wounds, wherein α-lactalbumin, a protein rich in tryptophan, has been demonstrated to possess healing properties in various studies, whereas the use of whey-extracted α-lactalbumin as a wound dressing has not yet been explored. In this study, the combination of lignin and α-lactalbumin was demonstrated to be efficacious in promoting wound healing in Swiss albino mice. Moreover, the lignin and α-lactalbumin combination was found to reduce scarring and hasten re-epithelization at a faster rate than the marketed silver sulfadiazine cream. Notably, the animals treated with α-lactalbumin loaded film exhibited greater stimulation in hair follicles and experienced hair regrowth earlier than their control counterparts. These findings suggest that the combination of lignin and α-lactalbumin holds promise as an effective wound dressing for promoting wound healing and reducing scarring.

In an embodiment of the present disclosure, there is provided development of a novel natural biocompatible multi-functional biopolymer herein used to further develop a prototype of a leather substitute, wherein the production of a prototype involved the complete adherence and sealing of both the leather substitute material and the leather reinforcement material. This was achieved by placing a weighty object on top of the materials for 48 hours at 25° C. and finishing the coating of wax emulsion, softener and wetting agents. The result was a fully sealed prototype that could then be cut to specifications, wherein a leather reinforcement material may be obtained from the fibres of coconut husk and/or coir pith powder, foam padding using EVA (ethylene-vinyl acetate), mesh fabrics like polyester or nylon, fiberglass, polyethylene or polycarbonate, rubber, and combinations thereof evenly attached to the cellulosic water-based adhesive present in the leather substitute material.

### MODE FOR INVENTION

### Examples

The working examples of the present disclosure would now be illustrated. These examples are neither restrictive nor intended to be limiting to the scope of the present invention. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein. It is to be understood that this disclosure is not limited to particular methods and experimental conditions described, as such methods and conditions may vary.

### Example 1

Extraction and Characterization of Lignin from Coconut Husk and/or Coir Pith Powder

### Materials

Coconuts and/or coir pith powder was purchased from various vendors in local market of Shirpur, Maharashtra, India; Sodium hydroxide, Sulfuric acid, Glutaraldehyde, other organic solvents and Starch were procured from Rankem, Mumbai, India; Xanthan Gum, Sorbitol, Glycerol were obtained from Loba Chemie Pvt. Ltd., Mumbai; HPC (Hydroxypropyl Cellulose) and PVA Hot (Polyvinyl Alcohol) were bought from Research Lab Pvt. Ltd., Mumbai. Coconut fibres were manually removed and husk was grinded in a kitchen grinder and passed through sieve No. 20 and 40 (IP; The Indian Pharmacopoeia) and powder was dried overnight in oven to remove excess moisture content and kept in sealed polybag at room temperature (25° C.) for further use.

Lignin Extraction Method from Coconut Husk Powder

Kraft pulping method was utilized for extraction of lignin from coconut husk powder. To extract lignin, two sample solutions of coconut husk powder, each in 200 ml of 12.5% w/v aq. NaOH containing dried coconut husk powder in the range of 10-80 g, more preferably in the range of 20-40 g, were made, and one solution was kept in an autoclave (Lequitron) at 130° C. for 3 hrs. and the other, in a microwave oven at 140° C. for 1 hr. (FIG. 3a), before further processing.

### Characterization of Lignin

### Yield of Extracts:

Extracted lignin weight/(coconut husk weight prior to lignin extraction) was used to determine lignin yield percentage. Coconut husk powder was dried in hot air oven at 60° C. for 12 hours to remove excess moisture. After alkaline treatment and lignin extraction through a muslin filter from dry pre-weighed coconut husk powder (W2), lignin in the filtered black liquor was subjected to precipitation by drop-wise addition of conc. sulfuric acid to pH 2. The precipitated lignin was separated and collected by either centrifugation or filtration on Whatman Paper (Grade 42, 2.5 μm pore size), dried in hot air oven at 60° C. for 8 hours, then washed twice with water to remove impurities, and dried again in hot air oven at 60° C. for 12 hours. Subsequently, the dried lignin was crushed using a mortar and pestle, and weighed again (W1).

$$Percentage yield = \frac{W 1}{W 2} \cdot 100$$

W1 is the weight of dried lignin powder obtained.

W2 is the initial weight of coconut husk powder taken.

With initial weight of 30 g husk powder (W1), approximately 8.4 g of lignin (W2) was obtained, resulting in 28% yield of lignin from coconut husk powder. The theoretical yield from various studies is around 34%, so percentage yield from Kraft extraction was around 82.35%. With improved heating method and pH optimization during extraction, this yield could be improved in future embodiments. Further micronisation of the coconut husk powder should also increase the yield.

### Particle Size and Zeta Potential

The particle sizes of lignin thus prepared and their surface charges were determined to evaluate the effect of pH on lignin particles obtained. For this experiment, 10 mg lignin was dissolved in 100 ml aqueous buffer solution at neutral (pH 7) and alkaline pH (pH 12), and using a syringe, the solutions were transferred to Zeta Sizer (Malvern) cuvette up to ¾th of its volume. Particle size and zeta potential were determined.

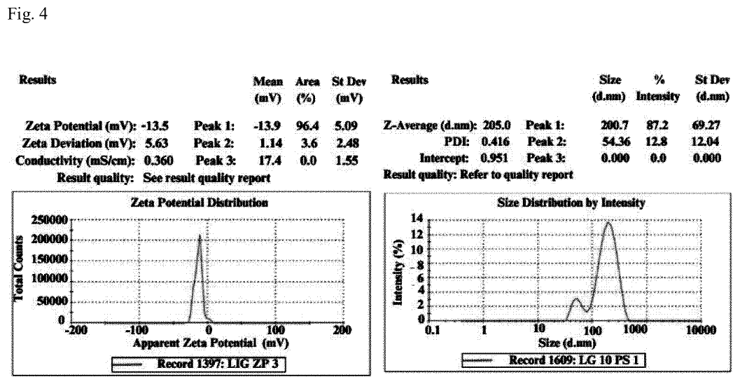

The objective of this study was to examine impact of pH on the size variation of lignin particles, depending on different pH ranges of solvents. It was found that the particle size of lignin decreases significantly as the pH increases from 5 to 13. This could be attributed to the fact that lignin was freely soluble in an alkaline solution (≥pH 12), and its isoelectric point was estimated to be pH 2. At neutral pH, the size of lignin particles was observed to be 323 nm, while for pH levels beyond 9, the particle size was reduced to 205 nm. At neutral pH, lignin exhibited a value of −13.5 mV (anionic), which falls within the stable range of −30 mV to +30 mV (FIG. 4).

### ATR-IR (Attenuated Total Reflectance-Infrared Spectroscopy) of Lignin

Lignin powder before analysis was dried in a hot air oven for 8 hours to remove moisture to reduce the noise in the results below. Lignin powder was crushed using a mortar and pestle and pressed under an ATR-IR sampler. The chemical components of the lignin were analyzed in the 4000 cm⁻¹ to 400 cm⁻¹ range using a Brookfield ATR-IR Spectrophotometer.

The FTIR (Fourier-transform infrared) spectrum provided shows the transmittance (%) of a lignin sample across a range of wavenumbers (cm⁻¹). The spectrum displays significant peaks that indicate the presence of functional groups in lignin. Specifically, there is a broad peak around 3400 cm¹, which corresponds to O—H stretching, and a sharp peak around 2930 cm⁻¹, indicative of C—H stretching. Additionally, characteristic peaks appear around 1600 cm⁻¹ and 1510 cm⁻¹, associated with aromatic C—C stretching, as well as a peak around 1260 cm⁻¹ corresponding to C—O stretching (aryl ether), and peaks near 1120 cm⁻¹ and 1030 cm⁻¹, related to guaiacyl units and C—O stretching in alcohols and ethers, respectively. (FIG. 5).

### X-Ray Diffraction (XRD)

To determine the nature of lignin powder and its purity with regard to amorphous to crystalline states, an X-ray diffractometer was used. The samples were crushed in a mortar and pestle, transferred to the cuvette, and then analyzed using Bruker D2 Phaser X-ray diffractometer. The voltage and current used were 40 kV and 30 mA, respectively, and the XRD patterns were obtained in the 20 range of 5° to 50° at a 0.02° step and 5°/min scan speed.

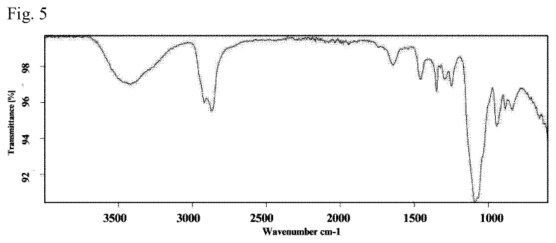

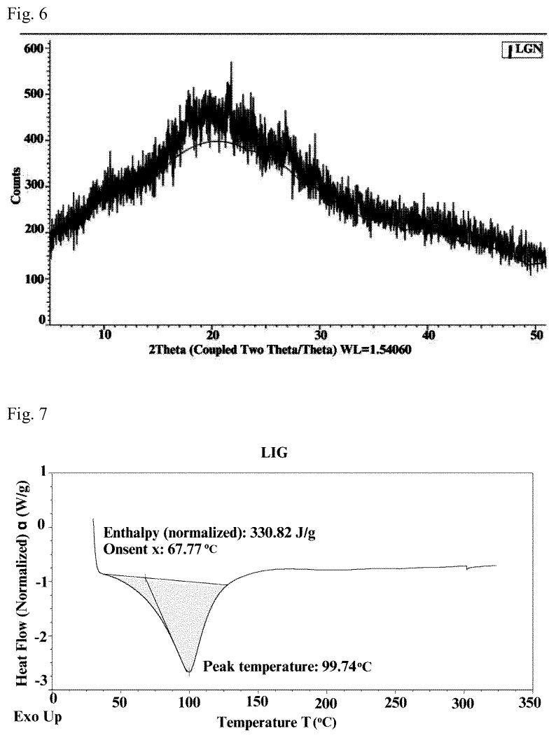

Extracted lignin had 86% amorphous nature and 14% crystallinity, lacking any sharp peaks. All the peaks remained in a relatively short range of 200 to 500 counts. Lignin typically exhibited a broad and diffuse scattering pattern. The amorphous nature of lignin arose from its complex and irregular polymeric structure. The phenolic monolignols formed a three-dimensional network lacking the regular repeating units and long-range orders that were characteristic of crystalline structures. The minor crystallinity was attributed to sulfuric impurities that were passed on from the lignin precipitation process and remained during the washing steps (FIG. 6).

### Differential Scanning Calorimetry

To evaluate the thermal characteristics of lignin, a Differential Scanning calorimeter (TA Instruments, DSC Discovery 250) was used to monitor the thermal events during the curing process. A 2.4 mg of lignin powder was thermally analyzed in the temperature range of 30° C. to 325° C. by placing it in an aluminum open pan. The DSC curve was recorded under N₂ atmosphere (50 mL/min) with a heating rate of 10° C./min.

The initial slope at 40° C. was attributed to instrumental calibration. Lignin showed an evident endothermic sharp peak due to the initial desorption of water content present in lignin, owing to the presence of moisture. Drying lignin powder at higher temperatures might have reduced moisture content but it could have affected the appearance of lignin as well as its solubility. The lack of any secondary peak showed that lignin was successfully extracted and isolated from cellulose, hemicellulose and xylan, as they degrade well in this range. Lignin did not show any phase change within the 300° C. range (FIG. 7).

### UV-Vis Spectrophotometer

The wavelength vs absorption spectrum was used to identify and determine the linearity of lignin. Using Shimadzu 1900 UV-Vis Spectrophotometer, 10 mg lignin was dissolved in 10 ml deionized water using a sonicator to obtain a stock solution of 1000 ppm and then diluted to 100 ppm. The λₘₐₓ value and the linearity range were determined.

In UV-Vis spectroscopy, lignin showed a peak at 272 nm (λₘₐₓ) quite common for Kraft lignin, originating from the aromatic rings/non-conjugated phenolic groups. Lignin has a typical absorption peak at both 230 nm and 275 nm due to the electron transition from non-conjugated phenolic structures in aromatics. Linearity was determined to be in the 20 μg/ml to 100 μg/ml concentration range in distilled water (R²=0.999).

### Miscellaneous Tests

Ferric Chloride test for phenol was done on a lignin 1% w/v aqueous solution by drop-wise addition of freshly prepared Ferric Chloride solution and the resultant solution was observed for visible color change. Polarized Light Microscopy (PLM) was used to understand particle shape, uniformity and size. Liquid solution of lignin in deionized water was observed under PLM at 40× and 100× magnification.

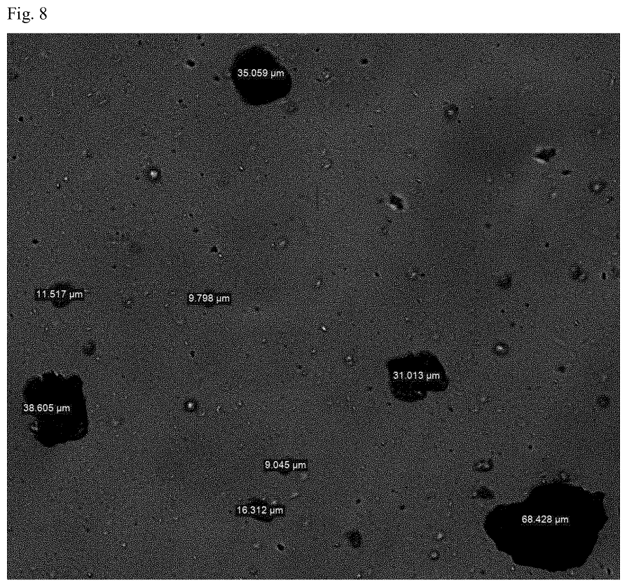

At neutral pH, the undissolved particles appeared in the range of 10 to 37 μm with few particles around 70 μm. Most of the dissolved lignin particles were in the range of 200-300 nm at neutral pH and 200 nm at pH 8 as seen in the Zeta Sizer. Particles exhibited uniformly circular to rhomboid shapes. Extracted lignin was dissolved in distilled water and the resulting solution gave a positive Ferric Chloride test by turning to deep green color (FIG. 8).

### Example 2

Pharmaceutical, Neutraceutical and Cosmetic Application of Extracted Lignin from Coconut Husk and/or Coir Pith Powder

### Formulation of Biopolymer

In the present invention the following approaches were taken to create three different biopolymers using different approaches.

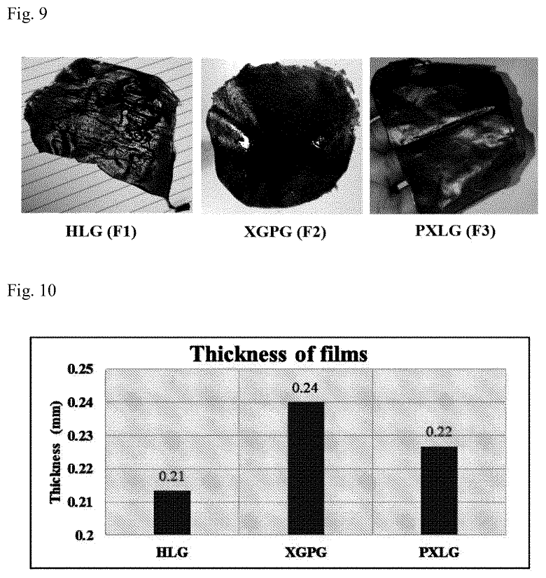

Using glutaraldehyde at a concentration of 2.5% as a cross-linker, a free-flowing solution was obtained by mixing HPC and lignin at a ratio of 3:2. Sulfuric acid was added drop-wise during mixing. The resulting solution was named HLG biopolymer (F1). A thick viscous solution, coded as XGPG biopolymer (F2), was obtained by mixing polyethylene glycol at a concentration of 10% with xanthan gum and lignin in a 1:1 ratio. The mixture was stirred at 2000 rpm; at 85-90° C. (High speed homogenizer, IKA T25 Digital Ultra Turrax, Mumbai, India). A thick solution, named PXLG biopolymer (F3), was obtained by mixing polyvinyl alcohol, xanthan gum and lignin in a 1:1:1 ratio; the mixture was stirred at 2000 rpm and at 85-90° C. with glycerol (1.5%) as a plasticizer (FIG. 9).

Subsequently, α-lactalbumin (ALA) extracted from whey protein as described in embodiments was added to each of the three biopolymers, and the solutions were mixed. Finally, the solutions were cast onto a silica mold (15×15 cm) and left to dry for 48 hours at 30° C. These biopolymers have the potential to revolutionize several fields, including medicine, food, packaging and leather substitution. Further research is required to explore their properties and potential applications in these and other fields.

### Example 3

### Characterization of Lignin Based Biopolymer

### Thickness

The width or thickness of the films was measured by randomly selecting five samples of similar size from each film and then measured 3 times each using the Mitutoyo Digimatic Caliper by carefully clasping the lower jaw to fit in the films. Special care was taken to avoid excess pressure while fitting the jaw of the caliper and after various readings, the mean value was calculated.

The thickness of films should not be too thick to feel intrusive for the patient while having enough depth to effectively protect the wound and contain it. For this purpose, the mean value of the thickness of films was calculated by taking 3 readings; XGPG film was the thickest at 0.24±0.01 mm, PXLG in the middle at 0.22±0.01 mm and thinnest HLG at 0.21±0.01 mm (FIG. 10).

### Foldability

To determine the foldability of films, five samples of each film were cut into 3×3 cm sizes held with clippers at the centre and pressed manually to fold and then unfolded, this process was repeated multiple times until the films showed visible cracks. The readings were noted and the mean value was calculated.

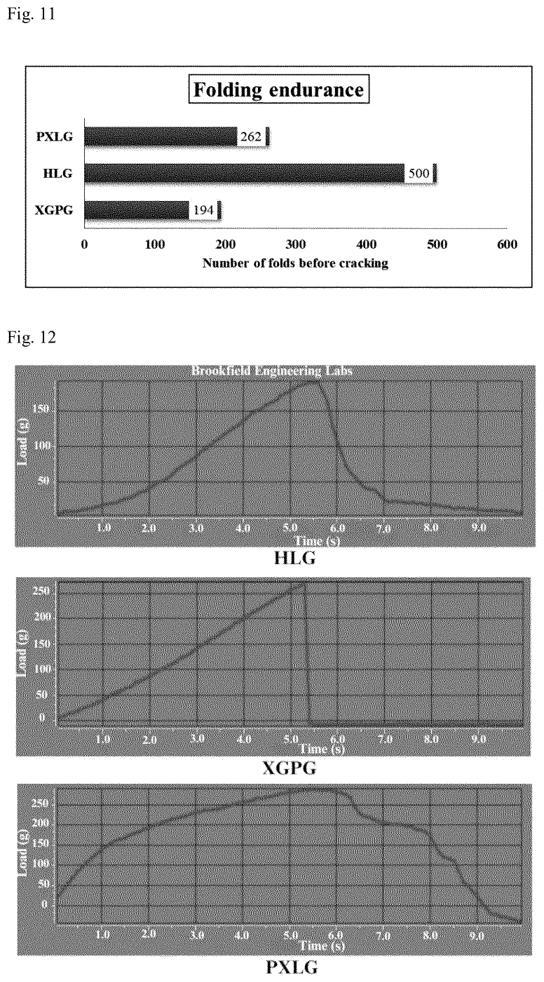

Any dressing designed for wound dressing must possess good folding endurance as it may be folded numerous times based on the location of the wound. For this reason, the folding capacity of films was evaluated and out of all the films, HLG had the best folding endurance and showed no cracks even after 500 folds while PXLG showed minor bends and then cracks around 250 and 290 folds respectively. Film XGPG also showed considerable endurance but showed cracks at 190 folds. Using the mean values for each, the following chart was drawn (FIG. 11).

### Texture Analysis and Elastic Modulus

Three samples of each film were cut into dimensions of 3×1 cm and analyzed using CT3 Texture Analyzer Machine (Brookfield Engineering Lab) at 30° C. and 1 bar. Stress data, deformation at the breaking point and modulus of elasticity were determined using the data obtained from this study.

To establish the physical nature of films, the modulus of elasticity was determined using the force applied upon the film of known length, using the formula indicated below.

$$E = F \cdot L / ( A \cdot δ )$$

F is the force (load) applied (in Newtons, N),
L is the original length of the sample (in meters, m),
A is the cross-sectional area of the sample (in square meters, m²),
δ is the deformation (change in length) of the sample (in meters, m).

PXLG showed the highest tensile strength, followed by XGPG and HLG, with maximum load before breaking for PXLG, XGPG and HLG being 339 g, 270 g and 191 g, respectively. Film PXLG also showed the most deformation at breaking point, which was 4.97 mm, followed by HLG at 2.75 mm and XGPG at 2.65 mm (FIG. 12).

The ability to resist change in shape is very important for any wound dressing, as it should be able to maintain its shape. Any shrinkage or lengthening may expose the wound to the outside environment and cause infection. It is also related to the level of physical protection the dressing should be able to provide.

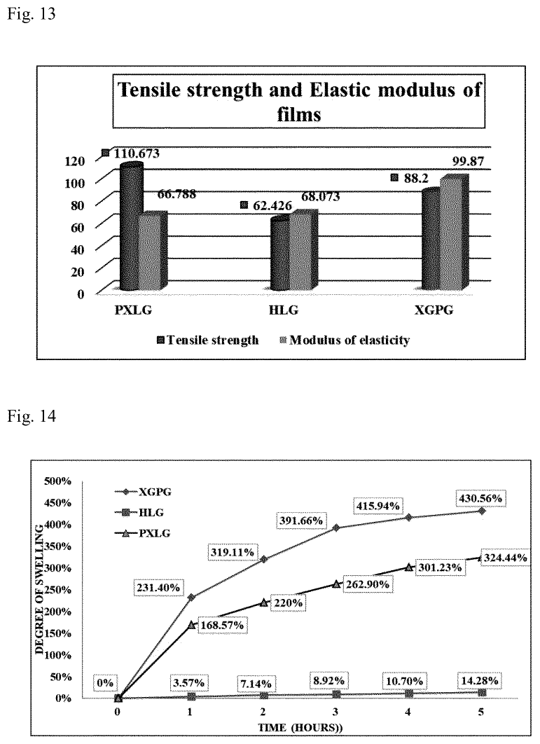

Converting load from grams to newton (1 gram-force is equal to 0.0098N) and using the formula mentioned before, Elastic modulus for PXLG, HLG and XGPG was determined as follows (FIG. 13).

Out of all the films, PXLG had the highest TS due to the addition of PVA and Glycerol-cross-linked-Xanthan gum (GCX). Further dehydration should also have increased its TS, as the stabilization of GCX and PVA would have been strengthened in lack of moisture. This was because the presence of water would have weakened the binding of hydroxyl group of glycerol with hydrophilic functional groups of xanthan gum. TS of XGPG films trailed behind PXLG due to the lower extent of crosslinking by PEG when compared to glycerol in the presence of PVA. While glycerol immobilizes, PEG remains mobile in the matrix (Domjan, Bajdik et al. 2009).

Film XGPG is significantly more rigid than both Film PXLG and Film HLG, while Films PXLG and HLG have comparable stiffness, with Film HLG being slightly stiffer than Film PXLG. Film XGPG is the stiffest, but Film PXLG is the strongest in terms of tensile strength. Film HLG is moderately stiff but has the lowest tensile strength. This correlation suggests that while stiffness (modulus of elasticity) and tensile strength can be related, they do not always increase proportionally. A material can be stiff but not necessarily strong, and vice versa.

### Degree of Swelling

The degree of swelling was determined by weighing (W1) 3 samples of each film of 1×1 cm size and then immersing them in beakers containing 15 ml phosphate buffer of pH 7.2 for 5 hours and weighing each sample every hour (W2). Using the formula indicated below, the degree of swelling was calculated.

$$Degree of swelling = \frac{W 2 - W 1}{W 1} \cdot 100$$

W1 is the initial weight of film.

W2 is the weight of film after dipping in water.

The degree of swelling is the measure of the degree of crosslinking as well as water absorption capacity without dissolving itself. The fraction that is not cross-linked shares the main role in swelling as such our theory that PEG acts as an external cross-linker was confirmed when XGPG had the highest swelling index after 5 hours at 430.56%, trailed by PXLG 324.44% due to Glycerol-cross-linked-Xanthan functional groups to make GCX which is more stable in waterless environment, with least swelling shown by HLG at 14.28% due to extensive cross-linking by glutaraldehyde between lignin and hydroxypropyl cellulose (FIG. 14).

### Water Retention

The dry weights of the films of size 2×2 cm were taken (W1) and then kept in a beaker having 50 ml water for 5 hours. After that, they were transferred to a centrifuge tube and centrifuged for 5 mins in a mini centrifuge (BioEra) for 5 minutes at 500 rpm. The supernatant was discarded and residues of the films were weighed again as wet weights (W2) as per the method mentioned by Wang et al. (2019)

The water retention value (WRT) of films was calculated using the formula indicated below.

$$WRT = \frac{W 2 - W 1}{W 2} \cdot 100$$

The ability to retain moisture is of prime importance for films aimed at wound dressing as wound exudes fluid and dressing must be able to withstand and contain that extracellular fluid. Moreover, it should be able to withstand water from outside without dissolving, to be suitable for practical use.

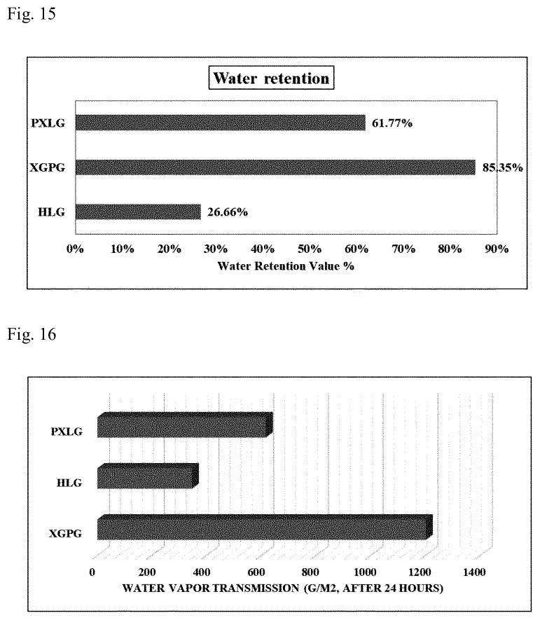

HLG showed hydrophobic tendencies and had the least capacity to retain water, while the other two films were able to retain 3 times more water than HLG, due to the presence of xanthan gum in the films (FIG. 15).

### Water Vapor Transmission

Three samples of each film were cut into sizes of 1×1 cm and attached using Teflon tape around the neck of a vial so that the films could completely block the vial opening of the vial with 5 mm radius containing 10 ml deionized water, without touching the water. The vial was left open to allow for free water vapour passage. Vials were weighed initially (W1) and weighed again after 12 hours (W2). Using the formula shown below, water vapour transmission was calculated for each film.

$$WVT = ( W 1 - W 2 ) / ( π {r}^{2} ) \cdot t$$

W1 is the initial weight of the vial with film and tape.

W2 is the weight of the vial with film and tape after 12 hours.

r is the radius of the vial opening.

t is the duration after which W2 was measured (i.e., 12 hours)

For a film to be used as dressing on wounds, it should be able to create a moist wound environment to help promote cell migration and wound healing. The ideal range is around 2000 g/m²·day. Out of all the films, HLG had the least vapour transmission, followed by PXLG. Both of these films had low vapour transmission due to better cross-linkage, whereas XGPG with PEG acting as an external cross-linker, its vapour transmission was nearly double when compared to the others (FIG. 16).

### ATR-IR

The samples were put under an ATR-IR sample holder and pressed slightly and then the samples were scanned in the range of 4000 cm⁻¹ to 400 cm⁻¹. The chemical components of the polymer films were analyzed through ATR-IR spectroscopy using a Brookfield ATR-IR Spectrophotometer.

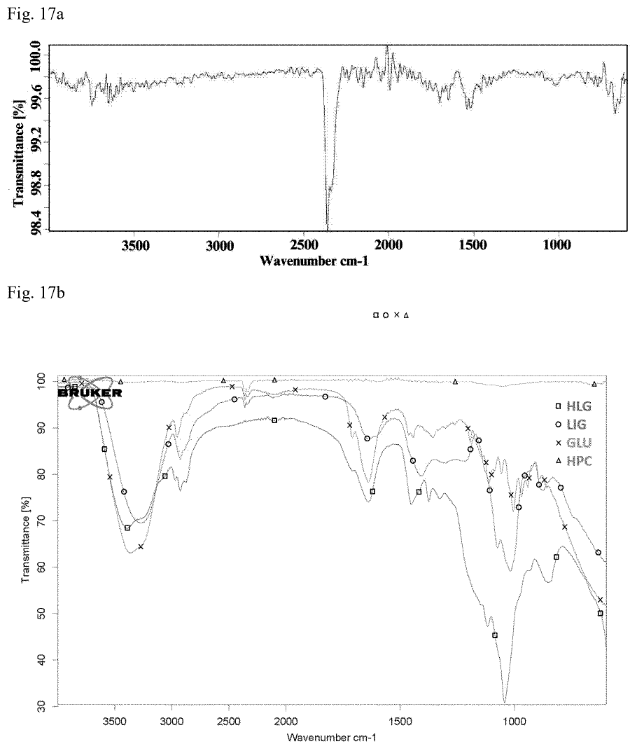

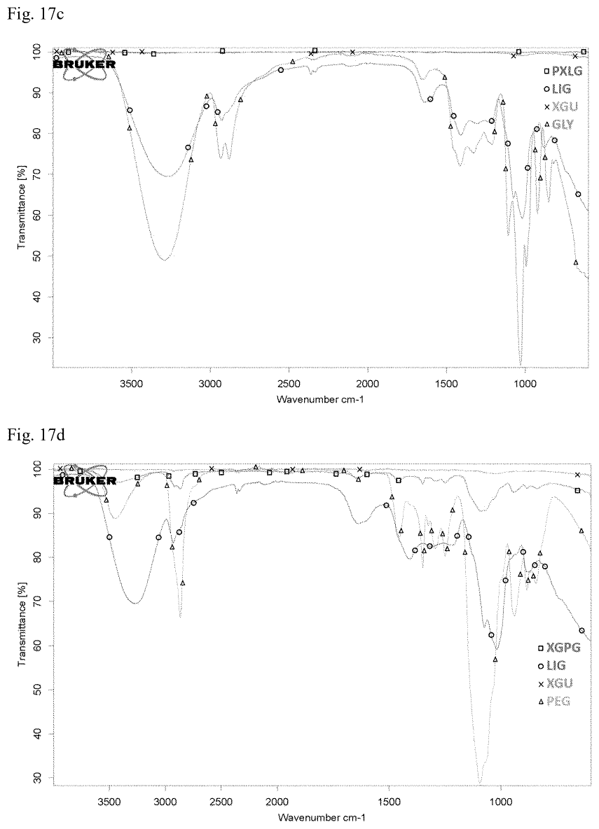

PEG 400 acts like an external cross-linker but glutaraldehyde showed the best chemical crosslinking with the introduction of new bonds in the form of hemiacetal and ester, while glycerol is known to impart flexibility with better tensile strength to the film at low concentrations (1-3% w/v) whereas sorbitol tends to increase flexibility drastically at high concentrations (8-10% w/v). These results also confirmed our theory that excess and unreacted glutaraldehyde can be removed easily by photolytic degradation (FIG. 17 a).

HLG shows a clear and broad peak in 3500 to 3200 cm⁻¹ due to the bound O—H group, and a peak at 2924 cm⁻¹ due to C—H stretching vibrations of methyl (CH3) present in the unsaturated aromatic system. Peak at 1462 cm⁻¹ was caused by C—H bending of the Methyl group. New peaks in the hemiacetal and ester band region of 1043 cm⁻¹ were due to C—O—C of acetal or hemiacetal groups and were considered the results of chemical cross-linkings (FIG. 17b).

Film PXLG showed an overlap in functional group spectra of lignin with xanthan gum and a reduction in the intensity or the disappearance of a peak associated with unbound O—H stretching vibrations indicates a decrease in free hydroxyl groups likely that the material has been chemically modified, reducing its interaction with water or tendency to absorb water. The split of the OH peak at 2950 cm⁻¹ is due to protons in the conjugated system. The extent of xanthan gum bound due to glycerol is evident as in a water-free environment, the degree of crosslinking is extensive enough to overshadow those with other compounds (FIG. 17c).

Film XGPG spectrum exhibited notable changes. The broad peak around 3200-3600 cm⁻¹ corresponding to O—H stretching vibrations showed a significant reduction in intensity, indicating the consumption of hydroxyl groups during cross-linking along with the other native peaks of lignin such as new peaks in the carbonyl and C—O stretching regions, and alterations in the aromatic ring vibrations, indicating the formation of new chemical bonds and structural changes in the lignin. The sharp peak at 2865 cm⁻¹ is due to —CH3 stretching which was similar to Marcos et al. (2017). Compared to PXLG, the lignin showed its characteristic peaks with less intensity; they were either engaged in crosslinking or present in less abundant than native lignin. This confirms that PEG acts as an external cross-linker and improves the stability of films by assisting the formation polymer network (FIG. 17d).

### XRD

The samples were cut into small square shapes and placed onto an XRD cuvette and analyzed using a Bruker D2 Phaser X-ray diffractometer. The voltage and current used were 40 kV and 30 mA, respectively, and the XRD patterns were obtained in the 2° range of 5° to 50° at a 0.02° step and 5°/min scan speed.

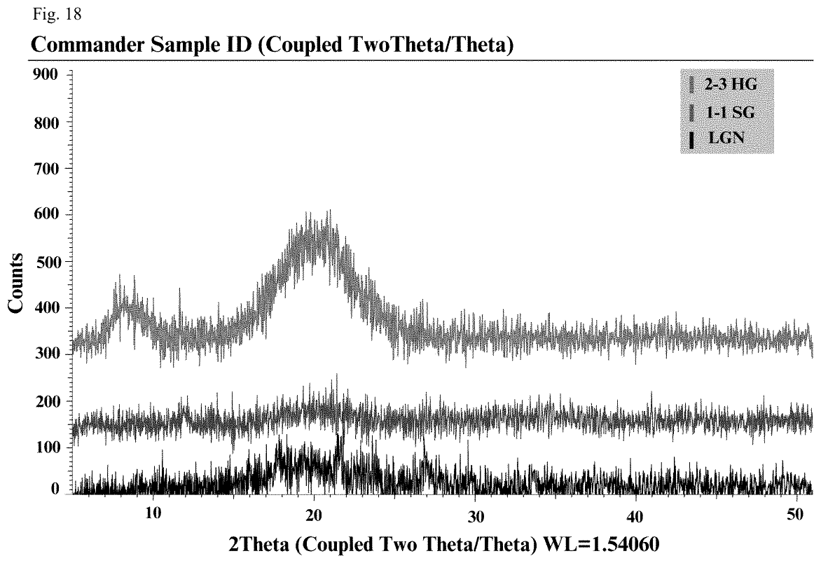

XRD analysis in 2° range of 5° to 50° showed films to be amorphous with some HPC peaks overshadowing Lignin. But overall these films remained amorphous. Films lacked crystalline peaks which showed the absence of crystalline impurities which might have incurred during the extraction process of Lignin (FIG. 18).

### DSC

XGPG film was loaded with alpha-lactalbumin and analyzed using a Differential Scanning calorimeter (TA Instruments, DSC Discovery 250) to understand its thermal properties during the curing process. A 2.3 mg of biopolymer film sample was analyzed in the temperature range of 30° C. to 325° C. by placing it in an aluminum open pan. The DSC curve was recorded under N₂ atmosphere (50 mL/min) with a heating rate of 10° C./min.

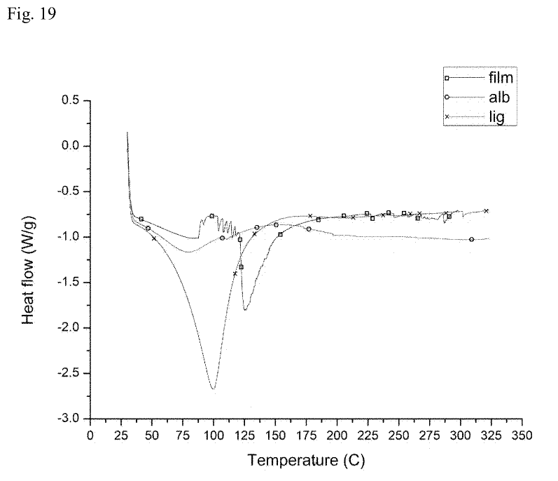

DSC studies revealed that film was able to protect α-lactalbumin from thermal degradation. The initial endothermic peak for alpha-lactalbumin in the range of 70 to 80° C. was assumed to be due to protein denaturation. However, in the film, this slope is slanted, signifying the protective nature of the film towards alpha-lactalbumin. The film also did not show any peak apart from one at 125° C. due to Xanthan gum present in the film. The exothermic peak for the film at 90 to 100° C. can be due to the glass transition of the blend (FIG. 19).

### SEM

The matrix of the film was analyzed by Scanning Electron Microscopy (JEOL JSM-6390 LA). SEM was employed to get a clear vision of the surface morphology and structure of the film. The SEM images of the surface of the polymer film were collected with SEM running under 200 Kv with a LaB6 Electron gun and the Point resolution was 0.23 nm.

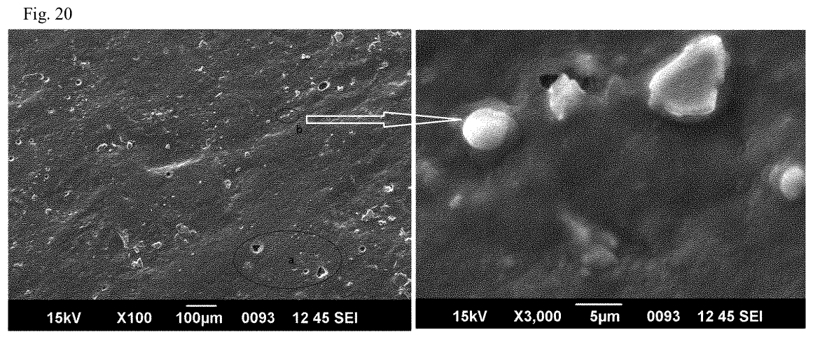

SEM Image showing the surface of XGPG films where the undissolved Lignin particles can be seen in (a) and (b) parts of the first image, but their occurrences are few, signifying that most of the lignin was linked with the xanthan gum film matrix (FIG. 20).

### Example 4

### In-Vivo Clinical Evaluation on Swiss Albino Mice

In this study, the combination of Lignin and α-lactalbumin was demonstrated to be efficacious in promoting wound healing in Swiss albino mice. Moreover, the Lignin and α-lactalbumin combination was found to reduce scarring and hasten re-epithelization at a faster rate than marketed silver sulfadiazine cream. Notably, the animals treated with α-lactalbumin loaded film exhibited greater stimulation in hair follicles and experienced hair regrowth before their counterparts. Wherein, α-lactalbumin (ALA), a protein commonly found in the milk of almost all mammalian species, plays a crucial role in controlling lactose production. In its multimeric form (Eugenia Lucena, Alvarez et al. 2006, 2007), α-lactalbumin strongly binds to calcium and zinc ions, suggesting potential bactericidal or antitumor effects (Sharma, Hanson et al. 2015). This affinity has been the focus of many studies to extract and isolate ALA (Eugenia Lucena, Alvarez et al. 2006, Eugenialucena, Alvarez et al. 2007), having wide range of activity, such as bactericidal, antitumor effects (Krunic, Rakin et al. 2018) and capable of wound healing (Guo, Liu et al. 2020). Among all the proteins, ALA has the highest percentage of tryptophan content. (Layman, Lonnerdal et al. 2018) Various studies and hypotheses have been proposed behind potential mechanisms of wound healing by exogenous tryptophan application including reducing cellular stress level by inhibiting indoleamine 2, 3-dioxygenase (IDO) and tumor necrosis factor-α (TNF-α) (Kiank, Zeden et al. 2010, Bandeira, Bortolot et al. 2015). Topical application of 1 μM Tryptophan has been previously demonstrated to reduce pain and increase re-epithelization in lower limb ulcer patients and has shown promise as wound healing natural moiety (Barouti, Mainetti et al. 2015). These findings suggested that the combination of lignin and α-lactalbumin held promise as an effective wound dressing for promoting wound healing and reducing scarring. Histopathological analysis of wound tissue samples revealed distinct differences among the groups. The Normal group displayed a well-preserved architecture of the epidermal, dermal, and subcutaneous layers, including intact sebaceous glands and hair follicles. In contrast, the Negative Control group exhibited significant alterations, such as hyperplasia, edema, and increased thickness of the epidermal layer. In addition, there was a mild infiltration of inflammatory cells, accompanied by acanthosis and hyperkeratosis.

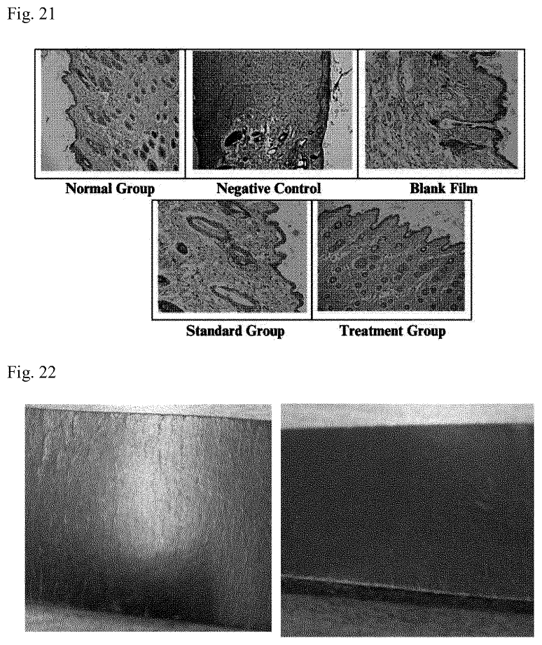

The Blank Film group maintained typical structures of the epidermis and dermis, indicating normal architecture, but, notably, lacked hair follicles. Both the Standard and Treatment groups demonstrated similar skin architecture in the epidermal and dermal layers. However, the Treatment group showed a markedly higher number of hair follicles compared to the Standard group, suggesting that α-lactalbumin may stimulate hair follicle growth. (FIG. 21).

(Normal group—no injury induced and kept for reference; Negative control was given injury but no treatment was given; Blank film group received blank lignin-based biopolymer film lacked alpha-lactalbumin; Standard group were also given injury was treated with marketed silver sulfadiazine cream: Treatment group were given injury and they were treated with films which contain alpha-lactalbumin).

### Example 5

### Leather Industry Application

Leather is an incredibly versatile material that finds use in a multitude of applications, including indoor furniture ornaments, clothing, footwear, luggage, handbags, accessories, and automotive applications. Conventionally, leather is derived from animal hides. However, a synthetic alternative has emerged, which involves coating natural or synthetic fibres with a synthetic polymer. Despite its popularity, the production of synthetic leather presents some environmental challenges, since it involves the use of non-biodegradable plastics, which are not only difficult to process but also take a considerable amount of time to decompose. Most synthetic leather is comprised of a polyester knit base, coated with polyurethane and polyvinyl chloride. Furthermore, while synthetic leather may be cheaper than natural leather, it is usually of lesser quality and durability.

A prototype of a leather substitute was manufactured using glutaraldehyde at 2.5% as a cross-linker at a lower ratio of HPC to Lignin (3:2) with dropwise addition of sulfuric acid, resulting in a free flowing solution, named HLG. Using Polyethylene glycol at 10% concentration for a 1:1 ratio between Xanthan gum and Lignin and stirring at high RPM at elevated temperature resulted in a thick viscous solution, coded XGPG. Polyvinyl alcohol, Xanthan gum and Lignin in a 1:1:1 ratio with glycerol at 1.5% concentration as a plasticizer and stirring at elevated temperatures at 2000 RPM resulted in a thick solution, named PXLG. All the solutions were then cast onto the silica mold respectively. A leather reinforcement material obtained from the fibres of coconut husk and/or coir pith powder was evenly attached to the cellulosic water-based adhesive present in the leather substitute material.

The production of a prototype involved the complete adherence and sealing of both the leather substitute material and the leather reinforcement material. This was achieved by placing a weighty object on top of the materials for 48 hours at standard room temperature and finishing the coating of wax emulsion, softener and wetting agents. The result was a fully sealed prototype that could then be cut to specifications.

The final product (as shown in the below FIG. 22—XGPG) exhibits excellent wear resistance and flexibility, particularly as a substitute for leather. Furthermore, it is unaffected by changes in weather and ultraviolet rays.

The modulus of elasticity was determined using the force acting upon the prototype finished leather of known length. The results are given in Table 1.

TABLE 1
Elastic modulus of lignin-based leather substitute.

| No. | Sample | Elasticity (10-100N) |
|---|---|---|
| 1 | HLG | 28.394 ± 2.46 |
| 2 | XGPG | 31.746 ± 1.72 |
| 3 | PXLG | 35.149 ± 2.91 |
| 4 | Standard leather | 45.782 ± 3.19 |

1. A natural biocompatible multi-functional biopolymer comprising;
a) Lignin extracted from the coconut husk and/or coir pith powder obtained from a fruit of the Cocos nucifera, a tropical plant that belongs to the Arecaceae (Palmae) family;
b) copolymers;
c) crosslinkers;
d) plasticizers; and
e) at least one active agent.

2. The natural biocompatible multi-functional biopolymer according to claim 1, wherein at least one copolymer is selected from either a natural polymer or a water-soluble synthetic polymer, wherein the natural polymers are such as hyaluronic acid and its derivatives, starch, modified starch, alginates, chitosan, chitin, natural gums, proteins such as gelatin, collagen, casein, zein, gluten, soy protein isolate, and whey protein isolate, and land plant extracts such as pectin, and combinations thereof, and wherein water-soluble synthetic copolymers include cross-linked polyacrylic acid chains, polyvinyl pyrrolidone, polyvinyl alcohol, modified cellulose ethers such as hydroxypropyl methylcellulose, hydroxyethyl cellulose, hydroxypropyl cellulose, carboxymethyl cellulose, and ion-exchange resins, and combinations thereof.

3. The natural biocompatible multi-functional biopolymer according to claim 1, wherein at least one crosslinker is selected from glutaraldehyde, formaldehyde, polyethylene glycol (PEG) and its derivatives, genipin, dextran, polyethyleneimine (PEI), ethylenediamine (EDA), and sugar alcohols/polyols like erythritol, xylitol, maltitol, mannitol, lactitol, polyglycitol, isomalt, sorbitol, glycerols, and combinations thereof, to promote crosslinking chemical reactions.

4. The natural biocompatible multi-functional biopolymer according to claim 1, wherein at least one plasticizer is selected from sucrose esters derivatives, glycerin, propylene glycol, polyethylene glycol, magnesium stearate, tallow, neatsfoot oil, lanolin, mineral oils, silicon oils, jojoba oils, castor oil, coconut oil, whale oil, squalene, grape seed oil, safflower oil, canola oil, almond oil, etc., and combinations thereof.

5. The natural biocompatible multi-functional biopolymer according to claim 1, wherein optionally an emulsifying agent is selected from xanthan gum, lanolin, sodium alginate, lecithin, casein, whey protein, pectin, ammonium phosphatides, acacia, and oleylamine.

6. The natural biocompatible multi-functional biopolymer according to claim 1, where it is used as a wound dressing or a part of the wound dressing on severe burn wounds of a patient to rejuvenate new tissue growths and replace damaged tissue on the burn wounds, wherein at least one active agent may be loaded from turmeric extract (curcumin), honey, green tea extract, gotu kola extract (Centella asiatica), α-lactalbumin, whey protein isolate, comfrey extract, tea tree oil, lavender oil, chamomile extract, calendula extract, aloe vera gel, silver sulfadiazine, bacitracin, hyaluronic acid, collagen, vitamin E, allantoin, sphingosine-/phytosphingosine-1-phosphate, and dexpanthenol (vitamin B5), and combinations thereof.

7. The natural biocompatible multi-functional biopolymer according to claim 1, where it is used as a wound dressing or a part of the wound dressing on severe burn wounds of a patient to rejuvenate new tissue growths and replace damaged tissue on the burn wounds, wherein at least one active agent may be loaded from human growth factors and/or cytokines such as Epidermal growth factor (EGF), Growth hormone/Somatotropin (hGH/STH), Vasoactive intestinal peptide (VIP), Fibroblast growth factor family proteins, i.e., Fibroblast growth factor-1 (FGF1), Fibroblast growth factor-2 (FGF2), Fibroblast growth factor-7/Keratinocyte growth factor (FGF7; KGF), Fibroblast growth factor-10/Keratinocyte growth factor-2 (FGF10; KGF2), Fibroblast growth factor-19 (FGF19), Fibroblast growth factor-21 (FGF21), Fibroblast growth factor-23 (FGF23), etc., Vascular endothelial growth factor (VEGF), Thymosin beta-4 (TMS), Stem cell factor/Steel factor (SCF), Platelet-derived growth factor (PDGF), Prolactin (PRL), Placental lactogen (hPL), Nerve growth factor (NGF), Brain-derived neurotrophic factor (BDNF), Glial cell-derived neurotrophic factor (GDNF), Ciliary neurotrophic factor (CNTF), Interleukin-1 receptor antagonist (IL1Ra), Interleukin family proteins, i.e., Interleukin-2 (IL-2), Interleukin-4 (IL-4), Interleukin-13 (IL-13), Interleukin-17 (IL-17), Interleukin-18 (IL-18), etc., Adiponectin (ApN), TSLP (Thymic stromal lymphopoietin), Bone morphogenetic protein family proteins, i.e., BMP-2, BMP-4, BMP-7, etc., Cluster of differentiation-34 (CD34), Desmoplakin (Dsp), Erythropoietin (EPO), Erythroid Differentiation Regulator 1 (ERDR1), Fibronectin, Follicle stimulating hormone (FSH), Luteinizing hormone (LH), human Chorionic gonadotropin (hCG), Follistatin (FST), Growth differentiation factor family proteins, i.e., Growth differentiation factor 11/Bone morphogenetic protein-11 (GDF11/BMP-11)), Growth differentiation factor 15 (GDF15), etc., Irisin, Kisspeptins, Klotho, Matrikines (extracellular matrix-derived peptides), Scube3 (signal peptide, CUB and EGF-like domain-containing protein 3), Osteopontin, Oxytocin, Pregnancy associated plasma protein-A (PAPP-A), Platelet factor 4 (PF4), Reelin, Stem cells antigen-1 (Sca-1), Stanniocalcin-1 (STC1), Stanniocalcin-2 (STC2), Transforming growth factor (TGF) family proteins, i.e., TGF-alpha, TGF beta-1, TGF beta-2, TGF beta-3, Thyrotropin-releasing hormone (TRH), Thyroid-stimulating hormone (TSH), Heat shock protein family proteins, i.e., HSP90A and its derivatives, etc., and their combinations thereof.

8. The natural biocompatible multi-functional biopolymer according to claim 1, wherein the combination of lignin and α-lactalbumin has demonstrated to make an efficacious wound dressings for severe burn wounds, wound healing, reducing scarring and hastening re-epithelization and to rejuvenate new tissue growths and replace damaged burn wound tissues.

9. The natural biocompatible multi-functional biopolymer according to claim 1, where it is used to develop a leather substitute or a part of the leather substitute, wherein a prototype of the leather substitute with or without using either α-lactalbumin or leather reinforcement materials obtained from the fibres of coconut husk and/or coir pith powder, exhibits excellent wear resistance and flexibility of those of the natural leather.

10. The natural biocompatible multi-functional biopolymer according to claim 6,
wherein the combination of lignin and α-lactalbumin has demonstrated to make an efficacious wound dressings for severe burn wounds, wound healing, reducing scarring and hastening re-epithelization and to rejuvenate new tissue growths and replace damaged burn wound tissues.

11. The natural biocompatible multi-functional biopolymer according to claim 7, wherein the combination of lignin and α-lactalbumin has demonstrated to make an efficacious wound dressings for severe burn wounds, wound healing, reducing scarring and hastening re-epithelization and to rejuvenate new tissue growths and replace damaged burn wound tissues.
